(19) United States
(12) Patent Application Publication
(10) Pub. No.: US 2012/0052466 A1
(43) Pub. Date: Mar. 1, 2012

(54) DENTAL ADHESIVE COMPOSITION COMPRISING ADHESION PROMOTING POLYMER ADDITIVE AND METHOD

(76) Inventors: Brian A. Shukla, Woodbury, MN (US); Sumita B. Mitra, West St. Paul, MN (US); Mahfuza B. Ali, Mendota Heights, MN (US)

(21) Appl. No.: 13/257,322

(22) PCT Filed: May 6, 2010
(86) PCT No.: PCT/US10/33872

§ 371 Date: Sep. 19, 2011

Related U.S. Application Data
(60) Provisional application No. 61/177,732, filed on May 13, 2009.

(51) Int. Cl. A61C 5/00; A61K 6/00

(52) U.S. Cl. 433/228.1; 525/288; 523/118

(57) ABSTRACT

Presently described are dental adhesive compositions and methods of use. The dental adhesive compositions described herein comprises a major amount of a hardenable component comprising a polymerizable organic component (i.e. having ethylenically unsaturated moieties) and a minor amount of a (e.g. water dispersible) polymer that comprises repeating units comprising polar or polarizable groups and repeating units comprising hydrophobic hydrocarbon groups.

### BACKGROUND

Various dental adhesive compositions and dental cements, that comprise a water dispersible polymeric film former, have been described.

Self-etching dental compositions, such as those described in U.S. Pat. No. 7,449,499, have also been described.

Saliva contamination is a pervasive problem is restorative dentistry. It is often challenging to maintain a clean, dry cavity preparation during the entire restorative process. This is particularly a problem is pediatric dentistry, where patient cooperation is highly variable. Ideally, isolation with a rubber dam is employed to prevent saliva contamination during a restorative procedure.

### SUMMARY

It has been found that the addition of a small concentration of a (e.g. water dispersible) polymer can improve the shear bond strength of a hardenable dental adhesive composition. The addition of such polymer can also improve the saliva contamination tolerance of the adhesive. Adhesives with improved saliva contamination tolerance, as described herein, would provide dentists with added assurance of successful clinical outcomes in situations when maintaining a clean, dry cavity preparation is difficult or impossible.

In one embodiment, a method of applying a dental composition is described. The method comprises providing a dental adhesive or primer composition comprising polymerizable organic component and an adhesion promoting polymer, the polymer comprising repeating units comprising one or more polar or polarizable groups and repeating units comprising one or more hydrophobic hydrocarbon group; applying the dental adhesive to a hard tissue surface; and hardening the polymerizable organic component. The hard dental tissue is typically enamel, dentin, or a combination thereof. In some embodiments, the hard dental tissue is wet, such as in the case of saliva contamination. In some embodiments, a (e.g. self-etching) adhesive is applied to the hard tissue surface without pretreating the surface with an etchant or primer. In other embodiments, such pretreatment may be employed.

In another embodiment, a (e.g. self-etching) dental adhesive composition is described comprising a polymerizable organic component comprising ethylenically unsaturated compound with acid functionality; and up to 10 wt-% of an adhesion promoting polymer, the polymer comprising repeating units comprising a polar or polarizable group and repeating units comprising a hydrophobic hydrocarbon group. The (e.g. self-etching) dental adhesive composition may optionally comprise a photocurable ionomer.

In another embodiment, a dental adhesive composition is described comprising a polymerizable organic component comprising at least one photocurable ionomer; and up to 10 wt-% of an adhesion promoting polymer, the polymer comprising repeating units comprising a polar or polarizable group and repeating units comprising a hydrophobic hydrocarbon group.

In each of these embodiments, the polymer typically comprises polar or polarizable groups derived from a carboxylic acid comprising no greater than 6 carbon atoms. The polymer preferably comprises ionic groups such as quaternary ammonium groups. The polymer typically comprises hydrophobic hydrocarbon groups comprising 8 to 22 carbon atoms. In a favored embodiment, the polymer comprises at least one acidic polymerizable group and one quarternary ammonium group. The hydrophobic group can be pendent from the polymer or be appended to the quarternary ammonium group. Additional modifying groups to modulate solubility and/or viscosity can be present in the polymer architecture.

The photocurable ionomer typically comprises pendent ionic groups and pendent free radically polymerizable groups, wherein at least one of said polymerizable groups is linked to said ionomer by means of an amide linkage. The ionomer preferably comprises carboxyl groups and (meth)acrylate groups. The adhesion promoting polymer typically lacks pendant ethylenically unsaturated moieties.

### DETAILED DESCRIPTION

As used herein, “adhesive” or “dental adhesive” refers to a composition used as a pre-treatment on a dental structure (e.g., a tooth) to adhere a “dental material” (e.g., “restorative,” an orthodontic appliance (e.g., bracket)) to a dental structure. Since a primer is also a pre-treatment, as used herein the term “adhesive” also includes primers to the extent such primers include an adhesion promoting polymer additive and a polymerizable component as described herein.

In some embodiments, the adhesive compositions are typically “unfilled” adhesive composition lacking a (e.g. inorganic oxide) filler. In this embodiment, the adhesive composition and polymerizable organic component are equivalent. In other embodiments, the “filled” adhesive composition comprises a polymerizable organic component and an (e.g. inorganic oxide) filler.

As used herein, “dental material” refers to a material that may be bonded to a dental structure surface and includes, for example, dental restoratives or orthodontic appliances.

In some embodiments, the dental structure surface can be pre-treated, e.g., by etching, priming, and/or applying an adhesive to enhance the adhesion with the dental material.

As used herein, an “etchant” refers to an acidic composition that is capable of fully or partially solubilizing (i.e., etching) a dental structure surface. The etching effect can be visible to the naked human eye and/or instrumentally detectable (e.g., by light microscopy). Typically, an etchant is applied to the dental structure surface for a period of about 10 to 30 seconds.

As used herein, a “self-etching” composition refers to a composition that bonds to a dental structure surface without pretreating the dental structure surface with an etchant. Preferably, a self-etching composition can also function as a self-primer wherein no primer is used.

As used herein, “hardening” or “curing” a composition are used interchangeably and refer to polymerization and/or crosslinking reactions including, for example, photo polymerization reactions and chemical polymerization techniques (e.g., ionic reactions or chemical reactions forming radicals effective to polymerize ethylenically unsaturated compounds) involving one or more materials included in the composition.

As used herein, “(meth)acryl” is a shorthand term referring to “acryl” and/or “methacryl.” For example, a “(meth)acryloxy” group is a shorthand term referring to either an acryloxy group (i.e., CH₂═CHC(O)O—) and/or a methacryloxy group (i.e., CH₂═C(CH₃)C(O)O—).

As used herein, a “hard tissue surface” refers to tooth structures (e.g., enamel, dentin, and cemented) and bone.

As used herein, a “wet” dental structure surface refers to a surface of a dental structure upon which an aqueous liquid (e.g., water or saliva) is present and visible to the naked human eye.

As used herein, a “dry” dental structure surface refers to a surface of a dental structure that has been dried (e.g., air dried) and does not have present visible water.

Presently described are dental adhesive compositions and methods of use. The dental adhesive compositions described herein comprises a major amount of a hardenable component comprising a polymerizable organic component (i.e. having ethylenically unsaturated moieties) and a minor amount of a (e.g. water dispersible) polymer that comprises repeating units comprising polar or polarizable groups and repeating units comprising hydrophobic hydrocarbon groups. Such polymer may be considered, and will also be referred to herein, as an adhesion promoting polymer additive.

The adhesion promoting polymer additive is generally present in the unfilled adhesive composition in an amount of at least 0.5 wt-%, 1 wt-% solids, 2 wt-% solids, or 3 wt-% solids. However, in some cases a higher concentration may also be necessary depending on the molecular weight of the said polymer. The polymer additive is generally present at the minimal amount to obtain the desired effect of increased (e.g. dry or saliva contaminated) bond strength. Hence, the amount of polymer additive is typically present in an amount no greater than about 8, 9, or 10 wt-% solids.

### Polar or Polarizable Groups

The adhesion promoting polymer additive comprises repeating units including a polar or polarizable group. Such polar or polarizable group are typically hydrophilic groups derived from vinylic monomers such as acrylates, methacrylates, crotonates, itaconates, and the like. The polar groups can be acidic, basic or salt. These groups can also be ionic or neutral. The polar or polarizable groups of the polymer generally render the polymer water-dispersible. Hence, such polymers have also been described as water-dispersible polymeric film-formers.

Examples of polar or polarizable groups include neutral groups such as hydroxy, thio, substituted and unsubstituted amido, cyclic ethers (such as oxanes, oxetanes, furans and pyrans), basic groups (such as phosphines and amines, including primary, secondary, tertiary amines), acidic groups (such as oxy acids, and thiooxyacids of C, S, P, B), ionic groups (such as quarternary ammonium, carboxylate salt, sulfonic acid salt and the like), and the precursors and protected forms of these groups. Additionally, a polar or polarizable group could be a macromonomer. More specific examples of monomers from which these polar or polarizable groups are derived from are described as follows.

Polar or polarizable groups may be derived from mono- or multifunctional carboxyl group containing molecules represented by the general formula:

CH₂═CR²G-(COOH)_{d}

where R²═H, methyl, ethyl, cyano, carboxy or carboxymethyl, d=1-5, and G is a bond or a hydrocarbyl radical linking group containing from 1-12 carbon atoms of valence d+1 and optionally substituted with and/or interrupted with a substituted or unsubstituted heteroatom (such as O, S, N and P). Optionally, this unit may be provided in its salt form. The preferred monomers in this class are monofunctional carboxylic acids having no greater than 6, 5, or 4 carbon atoms such as acrylic acid (AA), methacrylic acid, itaconic acid, and N-acryloyl glycine.

Polar or polarizable groups may, for example, be derived from mono- or multifunctional hydroxy group containing molecules represented by the general formula:

CH₂═CR²—CO-L-R³—(OH)_{d}

where R²═H, methyl, ethyl, cyano, carboxy or carboxyalkyl, L=O, NH, d=1-5 and R³ is a hydrocarbyl radical of valence d+1 containing from 1-12 carbon atoms. Suitable monomers in this class are hydroxyethyl(meth)acrylate, hydroxypropyl(meth)acrylate, hydroxybutyl(meth)acrylate, glycerol mono(meth)acrylate, tris(hydroxymethyl)ethane monoacrylate, pentaerythritol mono(meth)acrylate, N-hydroxymethyl(meth)acrylamide, hydroxyethyl(meth)acrylamide, and hydroxypropyl(meth)acrylamide.

Polar or polarizable groups may alternatively be derived from mono- or multifunctional amino group containing molecules of the general formula:

CH₂═CR²—CO-L-R³—(NR⁴R⁵)_{d}

where R², L, R³, and d are as defined above and R⁴ and R⁵ are H or alkyl groups of 1-12 carbon atoms or together they constitute a carbocyclic or heterocyclic group. Suitable monomers of this class are N-isopropyl acrylamide (e.g. NIPAAM); aminoethyl(meth)acrylate; aminopropyl(meth)acrylate; N,N-dimethylaminoethyl (meth)acrylate; N,N-diethylaminoethyl(meth)acrylate; N,N-dimethylaminopropyl (meth)acrylamide; N-isopropylaminopropyl(meth)acrylamide; and 4-methyl-1-acryloyl-piperazine.

Polar or polarizable groups may also be derived from alkoxy substituted (meth)acrylates or (meth)acrylamides such as methoxyethyl(meth)acrylate, 2-(2-ethoxyethoxy)ethyl(meth)acrylate, polyethylene glycol mono(meth)acrylate or polypropylene glycol mono(meth)acrylate.

Polar or polarizable groups units may be derived from substituted or unsubstituted ammonium monomers of the general formula:

CH₂═CR²—CO-L-R³—(N+R⁴R⁵R⁶)_{d}Q⁻

where R², R³, R⁴, R⁵, L and d are as defined above, and where R⁶ is H or alkyl of 1-12 carbon atoms and Q⁻ is an organic or inorganic anion. Suitable examples of such monomers include 2-N,N,N-trimethylammonium ethyl(meth)acrylate, 2-N,N,N-triethylammonium ethyl(meth)acrylate, 3-N,N,N-trimethylammonium propyl (meth)acrylate, N(2-N′,N′,N′-trimethylammonium)ethyl(meth)acrylamide, N-(dimethyl hydroxyethyl ammonium)propyl(meth)acrylamide, or combinations thereof, where the counterion may include fluoride, chloride, bromide, acetate, propionate, laurate, palmitate, stearate, or combinations thereof. The monomer can also be N,N-dimethyl diallyl ammonium salt of an organic or inorganic counterion.

Ammonium group containing polymers can also be prepared by using as the polar or polarizable group any of the amino group containing monomer described above, and acidifying the resultant polymers with organic or inorganic acid to a pH where the pendant amino groups are substantially protonated. Totally substituted ammonium group containing polymers may be prepared by alkylating the above described amino polymers with alkylating groups, the method being commonly known in the art as the Menschutkin reaction.

Polar or polarizable groups can also be derived from sulfonic acid group containing monomers, such as vinyl sulfonic acid, styrene sulfonic acid, 2-acrylamido-2-methyl propane sulfonic acid, allyloxybenzene sulfonic acid, and the like. Alternatively, polar or polarizable groups may be derived from phosphorous acid or boron acid group-containing monomers. These monomers may be used in the protonated acid form as monomers and the corresponding polymers obtained may be neutralized with an organic or inorganic base to give the salt form of the polymers.

Preferred repeating units of a polar or polarizable group include acrylic acid (AA), itaconic acid (ITA), N-isopropylacrylamide (NIPAAM), or combinations thereof. The polymer typically comprises at least 10, 15 or 20 wt-% of monomeric units derived from such polar or polarizable units.

### Hydrophobic Hydrocarbon Group

The adhesion promoting polymer additive also comprises repeating units including a hydrophobic hydrocarbon group. The hydrophobic hydrocarbon moiety typically comprises at least 8, 9 or 10 carbon atoms and typically no greater than 22 carbon atoms. The hydrocarbon moiety may be aromatic, yet is typically non-aromatic in nature, and optionally may contain partially or fully saturated rings. Preferred hydrophobic moieties are non-aromatic, long chain hydrocarbons typically having at least 12 carbons atoms such as lauryl (LA), dodecyl, octadecyl (ODA), and isooctyl (IOA) methacrylates. Such hydrophobic hydrocarbon moieties typically have a molecular weight of at least 160 g/mole.

In some embodiments, a polar or polarizable group and hydrophobic hydrocarbon group are derived from the same monomer. Hence, the same repeat unit comprises both a polar or polarizable group and a hydrophobic group. For example, when a substituted or unsubstituted ammonium monomers, as previously described, is employed and at least one of R⁴, R⁵, or R⁶ is an alkyl group having at least 8, 9, 10, 11, or 12 carbon atoms, such alkyl group is a hydrophobic hydrocarbon group. In this embodiment, at least one of R⁴, R⁵, or R⁶ may comprise up to 24 carbon atoms. A preferred monomer of this type is dimethylhexadecyl ammonium ethyl methacrylate bromide (DMAEMA-C₁₆Br).

Preferably, the adhesion promoting polymer additive comprises at least 3 or 5 wt-% of repeat units derived from a monomer comprising an ionic group and a hydrophobic group derived from an ammonium monomer such as dimethylhexadecyl ammonium ethyl methacrylate bromide. More preferably, the adhesion promoting additive comprises at least 10, 15 or 20 wt-% of such monomer. Generally, the adhesion promoting polymer additive comprises no greater than 40 wt-% of repeat units derived from a monomer comprising an ionic group and a hydrophobic group.

Other hydrophobic hydrocarbon moieties, such as those comprising a hydrophobic fluorine containing group, may also be employed as described in US2008/0305457; incorporated herein by reference.

The adhesion promoting polymer additive typically further comprises a modulating group. Exemplary modulating groups are derived from acrylate or methacrylate or other vinyl polymerizable starting monomers and optionally contain functionalities that modulate properties such as glass transition temperature, solubility in the carrier medium, hydrophilic-hydrophobic balance and the like.

Examples of modulating groups include the lower to intermediate methacrylic acid esters of 1-12 carbon straight, branched or cyclic alcohols. Typically the modulating groups have less than 8, 7 or 6 carbon atoms with C1-C4 being most typical. In some embodiments, the modulating group is derived from isobutyl methacrylate (IBMA).

In some embodiments, the adhesion promoting polymer additive is free of ethylenically unsaturated groups. The polymer may optionally include ethylenically unsaturated groups or other reactive groups such as epoxy groups or silane moieties.

In some embodiments, the adhesion promoting polymer additive also includes a repeating unit that includes a graft polysiloxane chain. The graft polysiloxane chain is derived from an ethylenically unsaturated preformed organosiloxane chain. The molecular weight of this unit is generally above 500 g/mole and can range up to 10,000 g/mole or greater.

Monomers used to provide the graft polysiloxane chain of this invention are terminally functional polymers having a single functional group (vinyl, ethylenically unsaturated, acryloyl, or methacryloyl group) and are sometimes termed silicone macromonomers or silicone macromers (SiMac). The inclusion of a repeating unit that includes a graft polysiloxane chain results in the polymer having silane moieties capable of undergoing a condensation reaction.

The requisite monomer(s) comprising polar or polarizable group(s) and hydrophobic hydrocarbon group(s), typically along with modulating monomer(s) and optional monomers as just described are polymerized to form a polymer. The weight average molecular weight of the polymer is typically at least 10,000 g/mole and at most 100,000 g/mole. In preferred embodiments, the adhesion promoting polymer additive is free of repeat units having a molecular weight above 500 g/mole.

Various polymers can be prepared as described for example in US2008/0305457; incorporated herein by reference. The following tables depict compositions of suitable exemplary adhesion promoting polymer additives.

| | | | Monomer | |
|---|---|---|---|---|
| | | | Comprising | |
| | | | Hydrophobic | Total |
| | | | Hydrocarbon | Monomer |
| | | | Moiety and | Providing |
| | | | Ionic Polar | Graft |
| Weight % Ratio | Modulating | Polar | Group (e.g. | Polysiloxane |
| of Monomeric | Monomer | Monomer | DMAEMA- | Chain |
| Units | (e.g. IBMA) | (e.g. AA) | C₁₆Br) | (e.g. SiMac) |
| Typical | 20-80 | 0-50 | 3-40 | 0-25 |
| Preferred | 60-80 | 10-40 | 10-30 | 0 |

| | | | Monomer | |
|---|---|---|---|---|
| | | | Comprising | |
| | | | Hydrophobic | Total |
| | | | Hydrocarbon | Hydrophobic |
| | | | Moiety and | Hydrocarbon |
| Weight % | | | Ionic Polar | Monomer |
| Ratio of | Modulating | Polar | Group (e.g. | (e.g. IOA |
| Monomeric | Monomer | Monomer | DMAEMA- | and/or |
| Units | (e.g. IBMA) | (e.g. AA) | C₁₆Br) | ODA) |
| Typical | 20-50 | 20-40 | 5-20 | 10-30 |

A minor amount of this (i.e. prepolymerized) polymer additive is combined with a conventional hardenable dental adhesive composition, comprising a polymerizable (e.g. (meth)acrylate) organic component, as known in the art.

In some embodiments, the adhesive composition is an aqueous dental adhesive comprising the polymerizable organic component and adhesion promoting polymer additive dispersed in water and a cosolvent such as ethanol or isopropyl alcohol. In such embodiments, the polymerizable organic component comprises water dispersible polymeric film formers.

In other embodiments, the adhesive composition is non-aqueous, including less than 1%, more preferably less than 0.5%, and most preferably less than 0.1% by weight water. In this embodiment, the polymerizable components of the adhesive composition need not be water dispersible.

Suitable photopolymerizable components that can be used in the dental compositions of the present invention include, for example, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof. Also suitable are polymerizable materials that contain both a cationically active functional group and a free radically active functional group in a single compound. Examples include epoxy-functional acrylates, epoxy-functional methacrylates, and combinations thereof.

In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable. In other embodiments, the compositions are chemically hardenable, i.e., the compositions contain a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements (e.g., conventional and resin-modified glass ionomer cements), redox cure systems, and combinations thereof.

Suitable hardenable compositions may include hardenable components (e.g., photopolymerizable compounds) that include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

The adhesive compositions described herein also typically include one or more hardenable components in the form of ethylenically unsaturated compounds without acid functionality.

Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate (TEGDMA), 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]ₚ-propoxyphenyldimethylmethane, ethoxylated bisphenolA di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.). Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.). Mixtures of two or more free radically polymerizable compounds can be used if desired.

The adhesive composition preferably comprises a hardenable component comprising a hydroxyl group(s) and ethylenically unsaturated group(s) in a single molecule. Examples of such materials include hydroxyalkyl (meth)acrylates, such as 2-hydroxyethyl (meth)acrylate (HEMA) and 2-hydroxypropyl (meth)acrylate; glycerol mono- or di-(meth)acrylate (e.g. GDMA); trimethylolpropane mono- or di-(meth)acrylate; pentaerythritol mono-, di-, and tri-(meth)acrylate; sorbitol mono-, di-, tri-, tetra-, or penta-(meth)acrylate, and 2,2-bis[4-(2-hydroxy-3-methacryloyloxy-propoxy)phenyl]propane (BisGMA). Suitable ethylenically unsaturated compounds are also available from a wide variety of commercial sources, such as Sigma-Aldrich, St. Louis, Mo. Mixtures of ethylenically unsaturated compounds can be used if desired.

The (e.g. self-etching) adhesive compositions described herein comprise one or more hardenable components in the form of ethylenically unsaturated compounds with acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, alpha, beta-unsaturated acidic compounds such as glycerol phosphate mono(meth)acrylates, glycerol phosphate di(meth)acrylates, hydroxyethyl (meth)acrylate phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate (MHP), (meth)acryloxyoctyl phosphate, bis((meth)acryloxyoctyl)phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Other ethylenically unsaturated compounds with acid functionality are known in the art such as described in previously cited US 2006/0204452.

The self-etching adhesive compositions comprise at least 5 wt-%, 10 wt-%, 15 wt-%, or 20 wt-% and no greater than 75 wt-% of ethylenically unsaturated compounds with acid functionality, based on the total weight solids of the unfilled composition. Ethylenically unsaturated compounds with acid functionality having at least one P—OH moiety, such as 6-methacryloxyhexyl phosphate, as typically preferred.

In some embodiments, the adhesive composition further comprises a photocurable ionomer, i.e. a polymer having pendent ionic groups capable of a setting reaction and pendent free radically polymerizable groups to enable the resulting mixture to be polymerized, i.e., cured, upon exposure to radiant energy.

As described for example in U.S. Pat. No. 5,130,347, photocurable ionomers have the general formula:

B(X)ₘ(Y)ₙ

wherein
B represents an organic backbone,
each X independently is an ionic group,
each Y independently is a photocurable group,
m is a number having an average value of 2 or more, and
n is a number having an average value of 1 or more.

Preferably the backbone B is an oligomeric or polymeric backbone of carbon-carbon bonds, optionally containing non-interfering substituents such as oxygen, nitrogen or sulfur heteroatoms. The term “non-interfering” as used herein refers to substituents or linking groups that do not unduly interfere with either the photocuring reaction of the photocurable ionomer.

Preferred X groups are acidic groups, with carboxyl groups being particularly preferred.

Suitable Y groups include, but are not limited to, polymerizable ethylenically unsaturated groups and polymerizable epoxy groups. Ethylenically unsaturated groups are preferred, especially those that can be polymerized by means of a free radical mechanism, examples of which are substituted and unsubstituted acrylates, methacrylates, alkenes and acrylamides.

X and Y groups can be linked to the backbone B directly or by means of any non-interfering organic linking group, such as substituted or unsubstituted alkyl, alkoxyalkyl, aryl, aryloxyalkyl, alkoxyaryl, aralkyl, or alkaryl groups.

Preferred photocurable ionomers are those in which each X is a carboxyl group and each Y is an ethylenically unsaturated group such as a (meth)acrylate group that can be polymerized by a free radical mechanism. Such ionomers are conveniently prepared by reacting a polyalkenoic acid (e.g., a polymer of formula B(X)ₘ₊ₙ wherein each X is a carboxyl group) with a coupling compound containing both an ethylenically unsaturated group and a group capable of reacting with a carboxylic acid group such as an NCO group. The resulting photocurable ionomer preferably has least one of the free radically polymerizable (e.g. (meth)acrylate group)) is linked to said ionomer by means of an amide linkage. The molecular weight of the resultant photocurable ionomers is typically between about 1000 and about 100,000 g/mole.

In some embodiments, that adhesive composition comprises hardenable components comprising hydroxyl groups(s) and ethylenically unsaturated group(s) in a single monomer. For example, the adhesive composition may comprise up to 20 to 40 wt-% of BisGMA and 15 to 35 wt-% of HEMA, 10 to 30 wt-% of GDMA. The total amount of such hardenable components is typically 70% to 80% wt-% solids of the cured adhesive composition. The adhesive may further comprises up to 10 wt-% of a urethane diacrylate (UDMA) and up to about 25 wt-% of photocurable ionomer.

In other embodiments, the (e.g. self-etching) adhesive composition comprises hardenable components comprising hydroxyl groups(s) and ethylenically unsaturated group(s) in a single monomer and hardenable components comprising acid functionality. For example, the adhesive composition may comprise up to 15 to 35 wt-% of BisGMA, 25 to 45 wt-% of HEMA, up to 5 wt-% of GDMA, and 15 to 35 wt-% of MHP. The adhesive may further comprise up to 10 wt-% of photocurable ionomer.

The cured adhesive compositions described herein typically include at least 50%, 60%, or 70% and at most 95% by weight solids derived from ethylenically unsaturated compounds, based on the total weight of the unfilled composition.

The adhesive composition may optionally contain a filler, which is for improving the coatability and the mechanical strength of the composition. For examples of the filler, referred to are those of inorganic fillers, organic fillers, inorganic/organic composite fillers.

One or more such fillers may be used either singly or as combined. Where the adhesive composition contains an organic solvent or water, the amount of the filler therein is preferably at most 30% by weight. Preferred fillers include colloidal silica and fumed silica having a mean particle size of at most 0.1 μm.

The adhesive composition in the invention may optionally contain polymerization inhibitors, antioxidants, UV absorbents, pigments dyes and other additives in addition to the ingredients noted above. A fluorine compound having anticarious capabilities, such as sodium fluoride, may be incorporated in the adhesive composition.

Methods of the present invention provide for the treatment of hard tissues, including human and animal tissues. Hard tissues include, for example, bone, teeth, and the component parts of teeth (e.g., enamel, dentin, and cementum).

When the dental adhesive compositions of the present invention include two or more parts, the two or more parts are preferably mixed just prior to or during the application process. Suitable mixing devices include, for example, static mixing devices.

The dental adhesive compositions are hardened, for example, by inducing the polymerizable organic components to react. For example, when the composition includes an ethylenically unsaturated group, polymerization may be induced by the application of actinic radiation. Preferably the composition is irradiated with radiation having a wavelength of 400 to 1200 nanometers, and more preferably with visible radiation. Visible light sources include, for example, the sun, lasers, metal vapor (e.g., sodium and mercury) lamps, incandescent lamps, halogen lamps, mercury arc lamps, fluorescent room light, flashlights, light emitting diodes, tungsten halogen lamps, and xenon flash lamps.

In some embodiments, the compositions are hardened (e.g., polymerized by conventional photo polymerization and/or chemical polymerization techniques) prior to applying the dental material. In other embodiments, the compositions are hardened (e.g., polymerized by conventional photo polymerization and/or chemical polymerization techniques) after applying the dental material. In some embodiments, the adhesive composition can promote adhesion to both enamel and dentin. Further, the composition can be formulated to function as the etchant, primer, and adhesive to both enamel and dentin.

Once the adhesive composition of the present invention has been hardened, the composition is generally not readily removed. Methods of bonding a dental material to a dental structure surface preferably result in a bond to enamel or dentin (or preferably both), of at least 10 MPa, more preferably at least 15, MPa, and most preferably at least 20 MPa when tested according to the Notched Edge Shear Adhesion test method described in the examples.

The present invention is illustrated by the following examples. It is to be understood that the particular examples, materials, amounts, and procedures are to be interpreted broadly in accordance with the scope and spirit of the invention as set forth herein. Unless otherwise indicated, all parts and percentages are on a weight basis, all water is deionized water, and all molecular weights are weight average molecular weight.

### EXAMPLES

### Test Methods

Preparation of Teeth: Bovine Incise Teeth, Free of Soft Tissue, were Embedded in Circular acrylic disks. The embedded teeth were stored in water in a refrigerator prior to use. In preparation for adhesive testing, the embedded teeth were ground to expose a flat enamel or dentin surface using 120-grit sandpaper mounted on a lapidary wheel. Further grinding and polishing of the tooth surface was done using 320-grit sandpaper on the lapidary wheel. The teeth were continuously rinsed with water during the grinding process. The polished teeth were stored in deionized water and used for testing within 8 hours after polishing. The teeth were allowed to warm in a 36° C. oven to between room temperature (23° C.) and 36° C. before use.

For the testing of dental adhesive compositions Control B and Examples 4-6 the adhesive was applied to dry or saliva coated surfaces without the use of an etchant.

For the testing of dental adhesive compositions Control A and Examples 1-3 the teeth were pretreated with an etchant, as described in Pretreatments I and II as follows, and then tested with the notched edge shear test method.

Pretreatment I: The 320-grit polished bovine enamel or dentin surface were air dried and then etched with etchant commercially available from 3M ESPE under the trade designation “Scotchbond” for 15 seconds. The etchant was rinsed off with water for 10 seconds and the tooth surface blotted dry leaving a slightly moist surface.

Preparation II: after Rinsing Off the Etchant and Blotting Dry the Tooth Surface as Described in Pretreatment I, a Microbrush Superfine Size disposable applicator was used to apply a layer of saliva to the entire bonding surface.

Notched Edge Shear Adhesion to Cut Enamel or Dentin

Two coats of the adhesive to be tested was applied (by painting with a brush) to the exposed flat enamel or dentin tooth surface, air dried for 5 seconds to evaporate the solvents and light cured for 10 seconds with a halogen curing device (commercially available from 3M ESPE under the trade designation “XL3000”. A 2 mm thick Teflon mold with a circular hole approximately 2.38 mm in diameter (such as commercially available from Ultradent) was clamped to the embedded tooth such that the hole in the mold exposed part of the adhesively prepared tooth surface. Curable composite material (commercially available from 3M ESPE under the trade designation “Filtek Z250”) was filled into the hole in the mold (such that the hole is completely filled, but not overfilled) and light cured with the halogen curing device, forming a cylindrical “button” that is adhesively attached to the tooth at one of the circular ends of the cylinder. The Teflon mold was carefully removed, leaving the composite button attached to the bonded tooth surface. The finished samples were stored in deionized water at 37° C. for approximately 24 hours prior to testing.

One at a time, the samples were mounted in an Instron machine such that the tooth surface was parallel to the direction of the pushing shear force. A metal fixture with a semicircular notched edge was attached to the Instron, and the notched edge was carefully fitted onto the button, flush with the tooth surface. The pushing shear force was started at a crosshead speed of 1 mm/min. The force in kg at which the bond failed was recorded, and this number was converted to a force per unit area using the known surface area of the button. The average bond strength of 4-5 samples was reported in megapascals (MPa).

### ABBREVIATIONS, DESCRIPTIONS, AND SOURCES OF MATERIALS

AA—Acrylic acid (Sigma-Aldrich)
IBMA—Isobutyl methacrylate (Sigma-Aldrich)
SiMac—Silicone macromer of MW about 10,000 (prepared as described for making “monomer C3b” at column 16 of U.S. Pat. No. 4,693,935 (Mazurek))
BisGMA—2,2-Bis[4-(2-hydroxy-3-methacryloyloxy-propoxy)phenyl]propane
HEMA—2-Hydroxyethyl methacrylate (Sigma-Aldrich, St. Louis, Mo.)
GDMA—Glycerol dimethacrylate
UDMA—Diurethane dimethacrylate (CAS No. 41137-60-4), commercially available as Rohamere 6661-0 (Rohm Tech, Inc., Malden, Mass.)
Photocurable ionomer—Example 11 of U.S. Pat. No. 5,130,347

### Photoinitiators

### CPQ—Camphorquinone (Sigma-Aldrich)

### EDMAB—Ethyl 4-(N,N-dimethylamino)benzoate (Sigma-Aldrich)

DPIHFP—Diphenyliodonium hexafluorophosphate (Johnson Matthey, Alpha Aesar

6-Hydroxyhexyl Methacrylate Synthesis: 1,6-Hexanediol (1000.00 g, 8.46 mol, Sigma-Aldrich) was placed in a 1-liter 3-neck flask equipped with a mechanical stirrer and a narrow tube blowing dry air into the flask. The solid idol was heated to 90° C., at which temperature all the solid melted. With continuous stirring, p-toluenesulfonic acid crystals (18.95 g, 0.11 mmol) followed by BHT (2.42 g, 0.011 mol) and methacrylic acid (728.49.02 g, 8.46 mol). Heating at 90° C. with stirring was continued for 5 hours during which time vacuum was applied using tap water aspirator for 5-10 minutes after each half-hour reaction time. The heat was turned off and the reaction mixture was cooled to room temperature. The viscous liquid obtained was washed with 10% aqueous sodium carbonate twice (2 times, 240 ml), followed by washing with water (2 times, 240 ml), and finally with 100 ml of saturated NaCl aqueous solution. The obtained oil was dried using anhydrous Na₂SO₄ then isolated by vacuum filtration to give 1067 g (67.70%) of 6-hydroxyhexyl methacrylate, a yellow oil. This desired product was formed along with 15-18% of 1,6-bis(methacryloyloxyhexane). Chemical characterization was by NMR analysis.

6-Methacryloxyhexyl Phosphate Synthesis: A slurry was formed by mixing P₂O₅ (178.66 g, 0.63 mol) and methylene chloride (500 ml) in a 1-liter flask equipped with a mechanical stirrer under N₂ atmosphere. The flask was cooled in an ice bath (0-5° C.) for 15 minutes. With continuous stirring, 6-hydroxyhexyl methacrylate (962.82 g, which contained 3.78 mol of the mono-methacrylate, along with its dimethacrylate by-product as described above) was added to the flask slowly over 2 hours. After complete addition, the mixture was stirred in the ice bath for 1 hour then at room temperature for 2 hours. BHT (500 mg) was added, and then the temperature was raised to reflux (40-41° C.) for 45 minutes. The heat was turned off and the mixture was allowed to cool to room temperature. The solvent was removed under vacuum to afford 1085 g (95.5%) of 6-Methacryloxyhexyl Phosphate (MHP) as a yellow oil. Chemical characterization was by NMR analysis.

### Starting Material Preparations

### Starting Material 1: Synthesis of Dimethylhexadecylammoniumethyl Methacrylate Bromide (DMAEMA-C₁₆Br)

A 500-ml round-bottom flask was charged with 42.2 parts of DMAEMA, 154.7 parts of acetone, 93.2 parts of 1-bromohexadecane (Sigma-Aldrich), and 0.34 parts of BHT. The mixture was stirred for 16 hours at 35° C. and then allowed to cool to room temperature. The resulting white solid precipitate was isolated by filtration, washed with cold ethyl acetate, and dried under vacuum at 40° C. An NMR analysis of the solid product revealed the structure to be pure dimethylhexadecylammoniumethyl methacrylate bromide.

### Polymer A: Preparation of Poly(IBMA(60)/AA(20)/DMAEMA-C₁₆Br(20))

IBMA (60 parts), AA (20 parts), DMA-C₁₆Br (20 parts), VAZO-67 (1.0 part), and isopropanol (300 parts) were combined in a reaction vessel and the resulting mixture purged with nitrogen for 2 minutes. The vessel was sealed and maintained at 60° C. in a constant temperature rotating device for 18 hours. The resulting clear viscous polymer solution was utilized in preparing the adhesive compositions of the present invention. Percent solids analysis revealed a quantitative conversion to polymer that was designated Polymer A and identified as the polymer of IBMA (60 parts), AA (20 parts), and DMA-C₁₆Br (20 parts), with weight ratios indicated in parentheses.

Polymers B and C were prepared as described for Polymer A and are listed as follows with monomeric units and weight ratios indicated:

Polymer B: IBMA (69 parts), AA (26 parts), DMAEMA-C₁₆Br (5 parts)

Polymer C: IBMA (64 parts), AA (20 parts), DMAEMA-C₁₆Br (12.5 parts) and SiMac (3.5 parts)

### Control A and Examples 1-3: Dental Adhesives Compositions

| | Control A | Example 1 | Example 2 | Example 3 |
|---|---|---|---|---|
| | (Wt % of | (Wt % of | (Wt % of | (Wt % of |
| | Component) | Component) | Component) | Component) |
| Component | Wt-% solids | Wt-% solids | Wt-% solids | Wt-% solids |
| BisGMA | (20.00) | (20.00) | (20.00) | (20.00) |
| | 33.3 | 31.7 | 31.7 | 31.7 |
| HEMA | (15.00) | (15.00) | (15.00) | (15.00) |
| | 25.0 | 23.8 | 23.8 | 23.8 |
| GDMA | (11.50) | (11.50) | (11.50) | (11.50) |
| | 19.2 | 18.3 | 18.3 | 18.3 |
| UDMA | (3.75) | (3.75) | (3.75) | (3.75) |
| | 6.3 | 6.0 | 6.0 | 6.0 |
| Photocurable | (8.75) | (8.75) | (8.75) | (8.75) |
| Ionomer | 14.6 | 13.9 | 13.9 | 13.9 |
| CPQ | (0.35) | (0.35) | (0.35) | (0.35) |
| | 0.58 | 0.56 | 0.56 | 0.56 |
| EDMAB | (0.25) | (0.25) | (0.25) | (0.25) |
| | 0.42 | 0.40 | 0.40 | 0.40 |
| DPIHFP | (0.40) | (0.40) | (0.40) | (0.40) |
| | 0.67 | 0.63 | 0.63 | 0.63 |
| deionized water | (5.00) | (5.00) | (5.00) | (5.00) |
| | 0 | 0 | 0 | 0 |
| ethanol | (35.00) | (27.68) | (27.68) | (27.00) |
| (200 proof) | 0 | 0 | 0 | 0 |
| Polymer B | | (7.32) | | |
| (41 wt-% | | 4.8 | | |
| solids in | | | | |
| ethanol) | | | | |
| Polymer A | | | (7.32) | |
| (41 wt-% | | | 4.8 | |
| solids in | | | | |
| ethanol) | | | | |
| Polymer C | | | | (8.00) |
| (37.5 wt-% | | | | 4.8 |
| solids in | | | | |
| ethanol) | | | | |

### Dental Adhesives Compositions A and Examples 1-3 Applied to Etched Enamel

| | | | Saliva | | |
|---|---|---|---|---|---|
| | | | contaminated, cut | | Change |
| Dental | Dry, cut enamel | | enamel (Prep II) | | between dry |
| Adhesive | (Prep I) | | (change vs control) | | and saliva |
| Composition | Avg | Std. Dev. | Avg | Std. Dev. | contaminated |
| Control A | 29.35 | ±3.28 | 22.59 | ±3.85 | −6.76 |
| Example 1 | 30.09 | ±5.62 | 23.86 | ±5.33 | −6.23 |
| | | | (+1.27) | | |
| Example 2 | 28.07 | ±3.42 | 27.32 | ±6.23 | −0.75 |
| | | | (+4.73) | | |
| Example 3 | 26.12 | ±3.25 | 23.12 | ±2.51 | −3.00 |
| | | | (+0.53) | | |

### Dental Adhesives Compositions A and Examples 1-3 Applied to Etched Dentin

| | | | Saliva | | |
|---|---|---|---|---|---|
| | | | contaminated, | | Change |
| Dental | Dry, dentin | | dentin (Prep II) | | between dry |
| Adhesive | (Prep I) | | (change vs control) | | and saliva |
| Composition | Avg | Std. Dev. | Avg | Std. Dev. | contaminated |
| Control A | 30.61 | ±5.55 | 22.90 | ±6.31 | −7.71 |
| Example 1 | 25.99 | ±6.66 | 19.19 | ±11.75 | −6.80 |
| | | | (−3.71) | | |
| Example 2 | 27.63 | ±4.52 | 28.99 | ±3.44 | +1.36 |
| | | | (+6.09) | | |
| Example 3 | 28.83 | ±3.31 | 29.77 | ±4.73 | +.94 |
| | | | (+6.87) | | |

### Control B and Examples 4-6: Self-Etching Dental Adhesives Compositions

| | Control B | Example 4 | Example 5 | Example 6 |
|---|---|---|---|---|
| | (Wt % of | (Wt % of | (Wt % of | (Wt % of |
| | Component) | Component) | Component) | Component) |
| Component | Wt-% solids | Wt-% solids | Wt-% solids | Wt-% solids |
| MHP | (17.8) | (17.1) | (17.1) | (17.1) |
| | 24.5 | 23.5 | 23.5 | 23.5 |
| BisGMA | (19.9) | (19.1) | (19.1) | (19.1) |
| | 27.4 | 26.2 | 26.2 | 26.2 |
| TEGDMA | (2.2) | (2.1) | (2.1) | (2.1) |
| | 3.0 | 2.9 | 2.9 | 2.9 |
| HEMA | (26.1) | (25.0) | (25.0) | (25.0) |
| | 36.0 | 34.3 | 34.3 | 34.3 |
| photocurable | (3.7) | (3.6) | (3.6) | (3.6) |
| ionomer | 5.1 | 4.9 | 4.9 | 4.9 |
| CPQ | (1.5) | (1.5) | (1.5) | (1.5) |
| | 2.1 | 2.1 | 2.1 | 2.1 |
| EDMAB | (1.0) | (1.0) | (1.0) | (1.0) |
| | 1.4 | 1.4 | 1.4 | 1.4 |
| DPIHFP | (0.4) | (0.4) | (0.4) | (0.4) |
| | 0.55 | 0.55 | 0.55 | 0.55 |
| deionized water | (12.5) | (12.4) | (12.4) | (12.4) |
| | 0 | 0 | 0 | 0 |
| ethanol (200 | (14.8) | (10.4) | (10.4) | (9.7) |
| proof) | 0 | 0 | 0 | 0 |
| Polymer B | | (7.3) | | |
| (41 wt-% | | 4.1 | | |
| solids in | | | | |
| ethanol) | | | | |
| Polymer A | | | (7.3) | |
| (41 wt-% | | | 4.1 | |
| solids in | | | | |
| ethanol) | | | | |
| Polymer C | | | | (8.0) |
| (37.5 wt-% | | | | 4.1 |
| solids in | | | | |
| ethanol) | | | | |

### Self-Etching Dental Adhesives Compositions B and Examples 4-6 Applied to Unetched Enamel

| | | | Saliva | | Change |
|---|---|---|---|---|---|
| Dental | Dry, cut enamel | | contaminated, cut | | between dry |
| Adhesive | (Change vs | | enamel | | and saliva |
| Composition | control) | | (Change vs control) | | contaminated |
| Control B | 19.97 | ±7.09 | 23.40 | ±1.57 | +3.43 |
| Example 4 | 26.00 | ±4.18 | 25.54 | ±4.37 | −0.46 |
| | (+6.03) | | | | |
| Example 5 | 25.52 | ±3.96 | 25.35 | ±1.84 | −0.17 |
| | (+5.55) | | | | |
| Example 6 | 27.99 | ±3.48 | 21.63 | ±3.28 | −6.36 |
| | (+8.02) | | | | |

### Self-Etching Dental Adhesives Compositions B and Examples 4-6 Applied to Unetched Dentin

| | | | Saliva | | Change |
|---|---|---|---|---|---|
| Dental | Dry, dentin | | contaminated, | | between |
| Adhesive | (Change vs | | dentin | | dry and saliva |
| Composition | control) | | (Change vs control) | | contaminated |
| Control B | 15.83 | ±10.07 | 23.85 | ±11.03 | +8.02 |
| Example 4 | 28.87 | ±13.60 | 35.30 | ±6.98 | +6.43 |
| | (+13.04) | | (+11.45) | | |
| Example 5 | 39.83 | ±6.96 | 41.13 | ±5.33 | +1.30 |
| | (+24.00) | | (+17.28) | | |
| Example 6 | 35.21 | ±6.66 | 26.95 | ±6.78 | −8.26 |
| | (+19.38) | | (+3.10) | | |

1. A method of applying a dental composition comprising:
providing a dental adhesive or primer composition comprising a polymerizable organic component and up to 10 wt-% solids of an adhesion promoting polymer, the polymer comprising:
repeating units comprising a polar or polarizable group; and
repeating units comprising a hydrophobic hydrocarbon group;
applying the dental adhesive to a hard tissue surface; and
hardening the polymerizable organic component.

2. The method of claim 1 wherein the hard dental tissue is selected from enamel, dentin, and a combination thereof.

3. The method of claim 1 wherein the surface of the hard dental tissue is wet.

4. The method of claim 1 wherein the adhesive is applied to the hard tissue surface without pretreating the surface with an etchant or primer.

5. The method of claim 1 wherein the adhesive is applied to the hard tissue surface with pretreating the surface with an etchant, primer, or combination thereof.

6. The method of claim 1 wherein the polymer comprises polar or polarizable group derived from one or more a carboxylic acids comprising no greater than 6 carbon atoms.

7. The method of claim 1 wherein the polymer comprises ionic groups.

8. The method of claim 7 wherein the ionic groups comprise quaternary ammonium groups.

9. The method of claim 1 wherein the polymer comprises hydrophobic hydrocarbon groups comprising 8 to 22 carbon atoms.

10. The method of claim 1 wherein the adhesive composition further comprises a photocurable ionomer.

11. The method of claim 10 wherein the photocurable ionomer comprises pendent ionic groups and pendent free radically polymerizable groups, wherein at least one of said polymerizable groups is linked to said ionomer by means of an amide linkage.

12. The method of claim 10 wherein the ionomer comprises carboxyl groups and (meth)acrylate groups.

13. The method of claim 1 wherein the adhesion promoting polymer lacks pendant ethylenically unsaturated moieties.

14. The method of claim 1 wherein the polymerizable organic component comprises at least one (meth)acryl monomer, (meth)acryl oligomer, (meth)acryl polymer, or mixture thereof.

15. The method of claim 1 wherein the dental adhesive composition further comprises an inorganic filler component.

16. A dental adhesive composition comprising
a polymerizable organic component comprising at least 10 wt-% of ethylenically unsaturated compound with acid functionality; and
up to 10 wt-% of an adhesion promoting polymer, the polymer comprising
repeating units comprising a polar or polarizable group, and
repeating units comprising a hydrophobic hydrocarbon group.

17. (canceled)

18. A dental adhesive composition comprising
a polymerizable organic component comprising at least one photocurable ionomer; and
up to 10 wt-% of an adhesion promoting polymer, the polymer comprising
repeating units comprising a polar or polarizable group, and
repeating units comprising a hydrophobic hydrocarbon group.

19. The dental adhesive composition of claim 18 wherein the photocurable ionomer comprises pendent ionic groups and pendent free radically polymerizable groups, wherein at least one of said polymerizable groups is linked to said ionomer by means of an amide linkage.

20-25. (canceled)

26. The dental adhesive composition of claim 16 wherein the polymerizable organic component comprises at least one (meth)acryl monomer, (meth)acryl oligomer, (meth)acryl polymer, or mixture thereof.

27. The dental adhesive composition of claim 16 wherein the dental adhesive composition further comprises an inorganic filler component.
